(19) United States
(12) Patent Application Publication
(10) Pub. No.: US 2006/0058859 A1
(43) Pub. Date: Mar. 16, 2006

(54) Cooling catheter and method with adjunctive therapy capability

(76) Inventor: Thomas Lad Merrill, East Windsor, NJ (US)

Correspondence Address: COOLEY GODWARD LLP;ATTN: PATENT GROUP, 11951 FREEDOM DRIVE, SUITE 1700, ONE FREEDOM SQUARE- RESTON TOWN CENTER, RESTON, VA, 20190-5061, US

(21) Appl. No.: 11/226,683

(22) Filed: Sep. 14, 2005

Related U.S. Application Data
(60) Provisional application No. 60/610,333, filed on Sep. 16, 2004.
(60) Provisional application No. 60/650,297, filed on Jan. 26, 2005.

(51) Int. Cl. A61F 7/00; A61F 7/12

(52) U.S. Cl. 607/105; 607/113

(57) ABSTRACT

A system and method for cooling blood is described. One embodiment includes a tube configured to be inserted into a blood vessel; a blood-conveyance pathway located inside the tube; a blood inlet configured to allow blood to enter the blood-conveyance pathway from the blood vessel; a blood outlet configured to allow blood to move from the blood-conveyance pathway into the blood vessel; a coolant-supply pathway located inside the tube and adjacent to the blood-conveyance pathway; a coolant-return pathway located inside the tube; and a coolant-turn-around connecting the coolant-supply pathway and the coolant-return pathway.

### PRIORITY

The present application claims priority from to commonly owned and assigned application No. 60/610,333 entitled Small Artery Cooling Catheter And Method With Adjunctive Therapy Capability, which is incorporated herein by reference. This application also claims priority to commonly owned and assigned application No. 60/650,297, entitled High Capacity Small Artery Cooling Catheter and Method with Adjunctive Therapy Capabilities, which is incorporated herein by reference.

### GOVERNMENT SUPPORT

The National Institute of Health provided support for the subject matter of this patent application under Grant # 1 R43NS049933-01A1 (An Active Mixing Catheter For Selective Organ Cooling) and the United States government may have rights in this application.

### COPYRIGHT

A portion of the disclosure of this patent document contains material that is subject to copyright protection. The copyright owner has no objection to the facsimile reproduction by anyone of the patent disclosure, as it appears in the Patent and Trademark Office patent files or records, but otherwise reserves all copyright rights whatsoever.

### FIELD OF THE INVENTION

The present invention relates generally to a medical device used to reduce tissue injury, including tissue injury resulting from ischemia, occurring naturally, through trauma, or from surgery. This invention, in some forms, also relates to the application of adjunctive therapies such as angioplasty and stent placement.

### BACKGROUND OF THE INVENTION

Experimental evidence has shown that reductions in tissue temperature can reduce the effects of ischemia or inadequate blood flow. Among other mechanisms, hypothermia decreases tissue metabolism, concentrations of toxic metabolic byproducts, and suppresses the inflammatory response in the aftermath of ischemic tissue injury. Depending on the time of initiation, hypothermia can be intra-ischemic, post-ischemic, or both. Hypothermic ischemic protection is preventive if tissue metabolism can be reduced. It may also enhance recovery by ameliorating secondary tissue injury or decreasing ischemic edema formation. Since the metabolic reduction is less than 10% per degree Centigrade, deep hypothermia targeting 20-25 degrees Centigrade, provides adequate tissue protection via metabolic slowdown. Secondary tissue injury, thought to be mainly caused by enzymatic activity, is greatly diminished by mild to moderate hypothermia targeting 32-35 degrees Centigrade. As early as 24 hours after onset of ischemia, secondary tissue injury can set off a mass effect with detrimental effects on viable surrounding tissues. Late post-ischemic hypothermia decreases edema formation and may therefore salvage tissue at risk.

To harness the therapeutic value of hypothermia the primary focus thus far has been on systemic body surface or vascular cooling. Systemic cooling has specific limitations and drawbacks related to its inherent unselective nature. Research has shown that systemic or whole body cooling may lead to cardiovascular irregularities such as reduced cardiac output and ventricular fibrillation, an increased risk of infection, and blood chemistry alterations.

Few concepts have attempted local, organ specific cooling. Local cooling approaches have been limited by the technological challenges related to developing tiny heat exchangers for small arterial vessels. These vessel inner diameters are 6 mm and smaller whereas larger systemic vessels are 20 mm or larger. The key advantage to localized or organ level cooling is the reduced thermal inertia, since the cooling capacity required is directly proportional to the mass being cooled. Cooling a portion of a 300 gram heart vs. 70,000 gram patient takes significantly less cooling capacity to reach equivalent reduced temperatures.

While hypothermia technologies have been progressing, the field of endovascular neurological intervention has also grown. Today therapeutic devices include stent placement, angioplasty, direct thrombolytic infusion, and mechanical devices for clot removal. In each of these therapeutic environments, ischemic damage is the focus. Boot-strapping local arterial based cooling together with these other emerging technologies will offer the patient optimal medical care. To accomplish this however, requires a cooling catheter system that not only cools effectively but also allows a pathway for adjunctive therapies using the endovascular tools mentioned above. Most of the embodiments of our cooling catheter address this challenge as well.

Heat transfer enhancement is the fundamental task for achieving safe, effective arterial cooling. It boils down to achieving the highest level of cooling capacity in the smallest volume possible. Heat exchanger design optimization attempts to achieve one or a combination of the following objectives: 1) reduce the size of the transport device, 2) increase the UA (U, the overall transport coefficient and A, the exchange surface area) to reduce the device—body fluid driving potential for exchange or increase the heat and or mass exchange rate, and 3) reduce the pumping power required to meet a heat and/or mass exchange target value. (Reference: Ralph L. Webb, Principles of Enhanced Heat Transfer, pg. 2, 1994).

Most related endovascular cooling catheter patents employ external passive transport enhancement techniques, where a fixed or static cooling catheter is placed inside a stagnant or moving body fluid. Passive techniques are transport enhancement approaches that do not add mixing energy to the fluid system of interest. The approach involves adding surface area and/or inducing turbulence adjacent to the effective exchange surface area. They are particularly effective when fluid pumping power is virtually limitless. In the human body, however, physiological constraints limit the hydraulic energy or fluid pumping power. As a result, passively enhanced devices in small arterial vessels are likely lead to substantial blood side flow resistance, diminishing organ perfusion levels.

In general, current designs are suited for the venous system, a system with large veins, significantly larger than small arteries. In this environment most of the devices have low heat exchange surface area to device volume ratios. This leads to potentially harmful vessel occlusion characteristics, particularly with smaller arterial blood vessels, increasing the chance of further ischemic injury. Unless additional energy is put into the blood flow stream, conservation of energy dictates that in most cases a boost in heat transfer will come at an increased cost in pressure drop. If the cardiovascular system cannot overcome this additional foreign resistance, perfusion rates must fall.

In general, the catheters do not have dedicated adjunctive therapy pathways. Again, the catheter designs are built largely for the venous applications where adjunctive therapies are less likely. As a result, these designs do not integrate well with existing endovascular tools, such as angioplasty catheters.

Although present devices are functional for venous applications, they are not sufficient for arterial applications. Accordingly, a system and method are needed to address the shortfalls of present technology and to provide other new and innovative features.

### SUMMARY OF THE INVENTION

Exemplary embodiments of the present invention that are shown in the drawings are summarized below. These and other embodiments are more fully described in the Detailed Description section. It is to be understood, however, that there is no intention to limit the invention to the forms described in this Summary of the Invention or in the Detailed Description. One skilled in the art can recognize that there are numerous modifications, equivalents and alternative constructions that fall within the spirit and scope of the invention as expressed in the claims.

There are several embodiments of the small artery cooling catheter. Some embodiments comprise an exchange catheter with heat and mass exchange surfaces, a transport catheter to carry the coolant, and a rear external hub used to connect the device to an outside control console and engage adjunctive therapeutic devices. One particular embodiment uses natural pressure differences between the aorta and the end organ to carry blood inside the cooling catheter. Cooling in the preferred embodiment occurs as blood contacts cold surfaces. Another embodiment is a hybrid surface-infusion cooling device. Coolant infusion enhances cooling catheter blood flow performance in two ways: 1) by reducing near-wall viscosity and making the inner catheter walls more slippery and 2) exchanging momentum with the blood. Additional embodiments use external surfaces to cool blood. Still additional embodiments use blood shuttling to boost heat transfer effectiveness and reduce device size.

As previously stated, the above-described embodiments and implementations are for illustration purposes only. Numerous other embodiments, implementations, and details of the invention are easily recognized by those of skill in the art from the following descriptions and claims.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawings wherein.

In the drawings, closely related figures have the same number but different alphabetic suffixes.

FIG. 1 shows a schematic of the cardiovascular network of the head and neck.

FIG. 2 shows a schematic of the carotid artery.

FIG. 3 shows a drawing of the coronary arteries on the heart.

FIG. 4 shows a 3D view of the coronary arteries at two different periods within the cardiac cycle.

FIG. 5 shows a drawing of the anatomy after heart bypass surgery.

FIG. 6 shows a schematic of a guide catheter engaged or coupled with the ostium of the coronary arteries.

FIG. 7 shows a graph of pressure and myocardial resistance, of the left ventricle, two factors that determine graft blood flow FIG. 8 shows a graph of graft blood flow rates to the left ventricle during a cardiac cycle.

FIG. 9 shows a graph of graft differential pressure and myocardial resistance of the right ventricle.

FIG. 10 shows a graph of graft blood flow rates to the right ventricle during a cardiac cycle.

FIG. 11 shows a graph of the Reynolds number of the blood flow to the common carotid artery.

FIG. 12 shows a graph of the flow rate distribution within carotid arteries.

FIG. 13 shows a graph of required cooling load as function of blood flow rate and blood temperature

FIG. 14 shows a side view of a preferred embodiment arterial cooling catheter.

FIG. 15 shows a catheter profile or cross-section of the preferred embodiment

FIG. 16 shows a catheter profile of another embodiment.

FIG. 17 shows a catheter profile of another embodiment.

FIG. 18 shows a catheter profile of yet another embodiment.

FIG. 19 shows a drawing of how mandrels are used to support inner lumens during the braiding process.

FIG. 20 shows an isometric cut-away view of the distal tip of the preferred embodiment.

FIG. 21 shows a 3D solid view of the distal tip of the preferred embodiment.

FIG. 22 shows a cut-away view of the proximal hub or connector for the cooling catheter.

FIG. 23 shows a drawing of a proximal balloon located on the shaft of the cooling catheter.

FIG. 24 shows a drawing of a distal balloon located on the shaft of the cooling catheter.

FIG. 25 shows a schematic of blood entry into the cooling catheter

FIG. 26 shows a schematic of coolant entry into the cooling catheter

FIG. 27 shows another schematic of coolant entry into the cooling catheter

FIG. 28 shows another schematic of coolant entry into the cooling catheter together with a hydrodynamic boundary layer

FIG. 29 shows a cut-away isometric drawing of the cooling catheter showing the blood entry and coolant entry holes.

FIG. 30 shows another isometric drawing of the cooling catheter showing the blood entry and coolant entry holes.

FIG. 31 shows another cut-away isometric drawing of the cooling catheter showing the blood entry and coolant entry holes.

FIG. 32 shows a graph of hematocrit changes and cooling capacity at different infusion levels.

FIG. 33 shows a drawing of catheter profile that has undergone post-extrusion forming

FIG. 33A a multisided mandrel for increasing the inner lumen wall surface area

FIG. 34 shows the cooling catheter engaged within an existing standard guide catheter.

FIG. 35 shows a cross sectional view of the cooling catheter within a standard guide catheter.

FIG. 36 shows an isometric drawing of the distal tip of the cooling catheter that engages the standard guide catheter

FIG. 37 shows a cut-away isometric drawing of the distal tip of the cooling catheter that engages the standard guide catheter

FIG. 38 shows a drawing of a distal tip of the engaged cooling catheter

FIG. 39 shows a drawing of another distal tip of the engaged cooling catheter.

FIG. 40 shows a drawing of another distal tip of the engaged cooling catheter as the blood exits the device.

FIG. 41 shows a drawing of another distal tip of the engaged cooling catheter as the blood enters the device.

FIG. 42 shows a drawing of a turbine balloon used to propel blood through the cooling catheter.

FIG. 43 shows a drawing of a wedge balloon used to propel blood through the cooling catheter.

FIG. 44 shows a drawing of a turbine balloon placed inside the cooling catheter.

FIG. 45 shows a drawing of an inflatable cooling catheter that allows for adjunctive therapy with continuous coolant flow.

FIG. 46 shows a profile drawing of a folded inflatable cooling catheter

FIG. 47 shows a profile drawing of the unfurled inflatable cooling catheter.

FIG. 48 shows an isometric drawing of the unfurled inflatable cooling catheter

FIG. 49 shows a 3D solid drawing of a heterogeneous compliant cooling catheter

FIG. 50 shows a profile drawing of blood shuttling cooling catheter.

FIG. 51 shows another profile drawing of blood shuttling cooling catheter.

FIG. 52 shows an isometric view of a blood shuttling cooling catheter.

### DETAILED DESCRIPTION

Referring now to the drawings, where like or similar elements are designated with identical reference numerals throughout the several views, and referring in particular to:

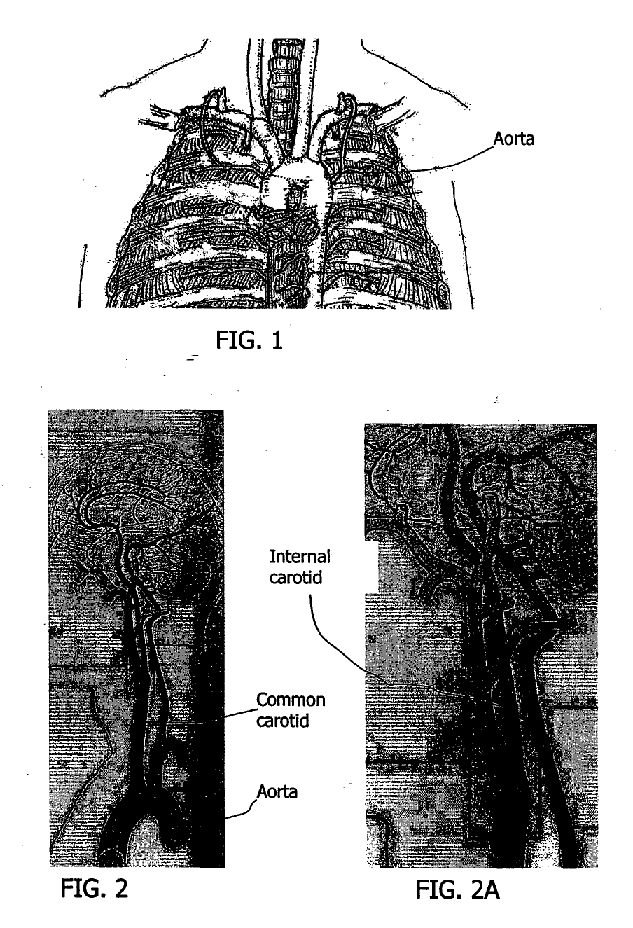

Description—FIGS. 1-4

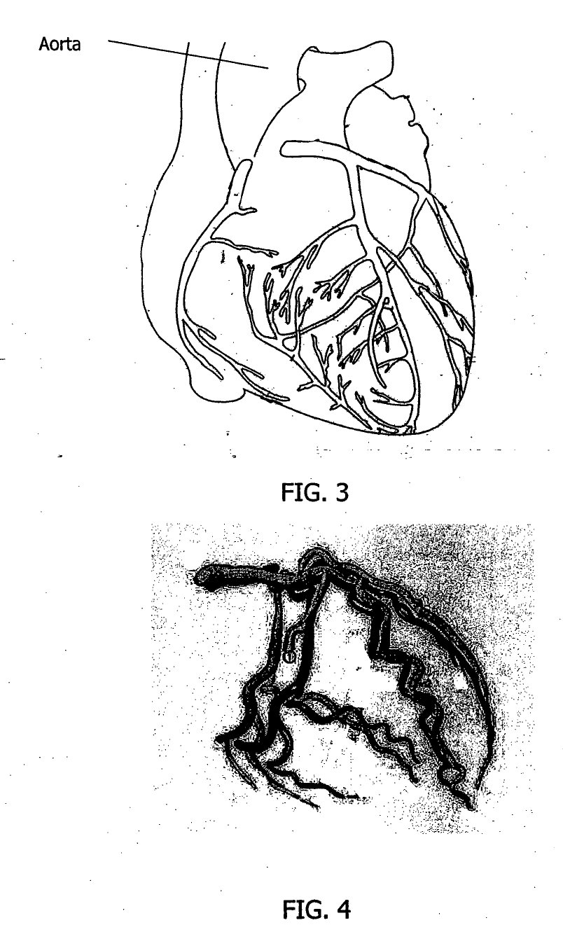

FIGS. 1 through 4 show the physiological landscape where embodiments of the present invention can be used. FIG. 1 shows a drawing of the aorta. The common carotid artery (FIG. 2) inner diameter ranges from 6 to 8 mm and its length ranges from 8 to 12 cm. The coronary arteries (FIGS. 3 and 4) are significantly smaller with proximal inner diameters ranging from 2 to 3.5 mm and length ranging from 2-4 cm. FIG. 4 shows the coronary artery spatial movement from start to stop during the cardiac cycle.

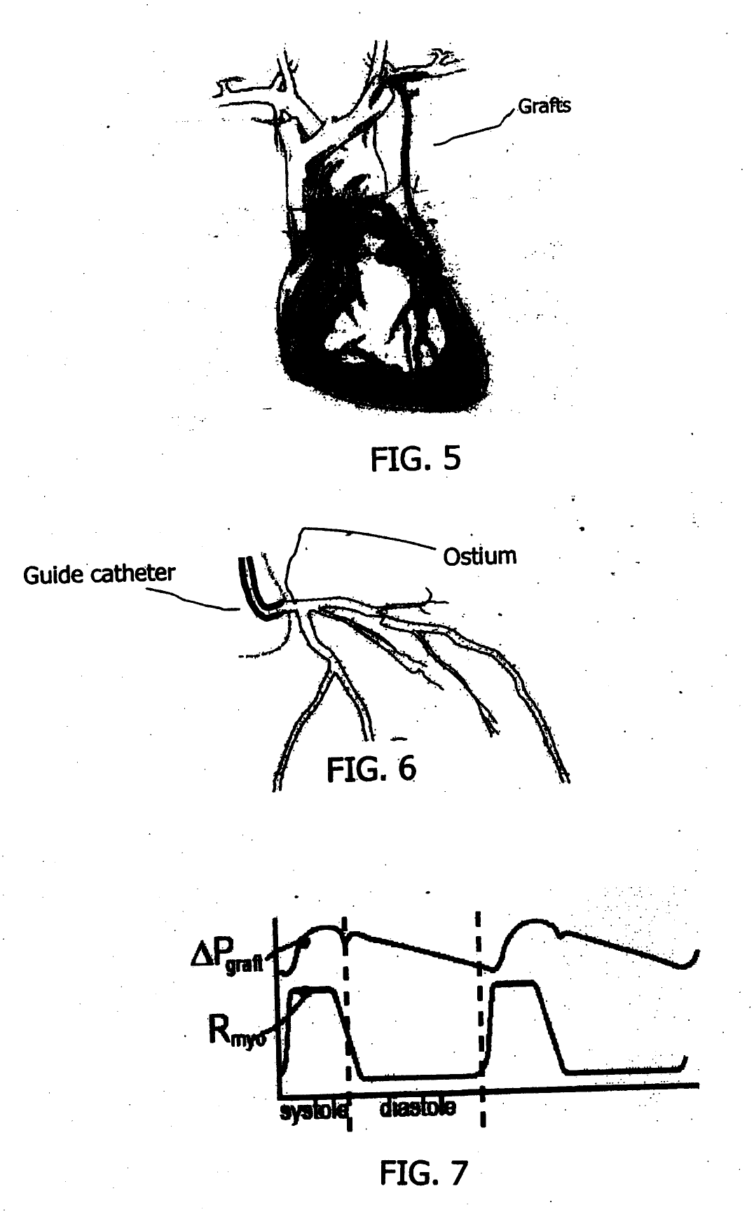

Description—FIGS. 5-13

FIG. 5 shows a drawing of the anatomical outcome of a bypass surgery. Grafts are analogous to highway bypasses, allowing blood flow past a coronary artery occlusion or blockage site. An embodiment that employs internal cooling embodiment uses an analogous approach to transfer cold blood to the heart.

FIG. 6 shows the typical engagement or insertion of a standard guide catheter (shown with thick black lines) at the coronary ostium, the entry point of the coronary artery. Through this guide catheter other interventional tools are passed such as dilation catheters and stents. The embodiment that employs internal cooling also engages the ostium in this manner.

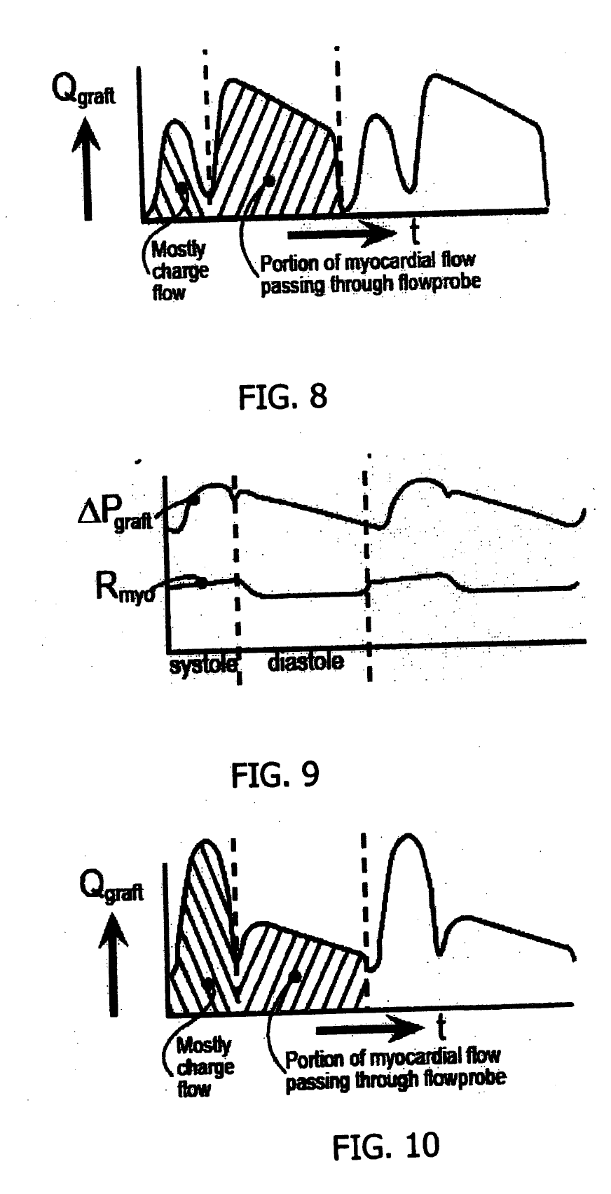

FIGS. 7-10 show graphs of the pressure, resistance, and flow behavior of a graft and the myocardium using a Transonic Inc., Ithaca, N.Y., transit-time ultrasound flow meter. FIGS. 7 and 8 show a graph of the left ventricle behavior while FIGS. 9 and 10 address the right ventricle. This same flow and pressure differential behavior takes place when the internal cooling catheter embodiment is used. The internal cooling catheter acts as bypass graft carrying cooled blood from the aorta to the heart.

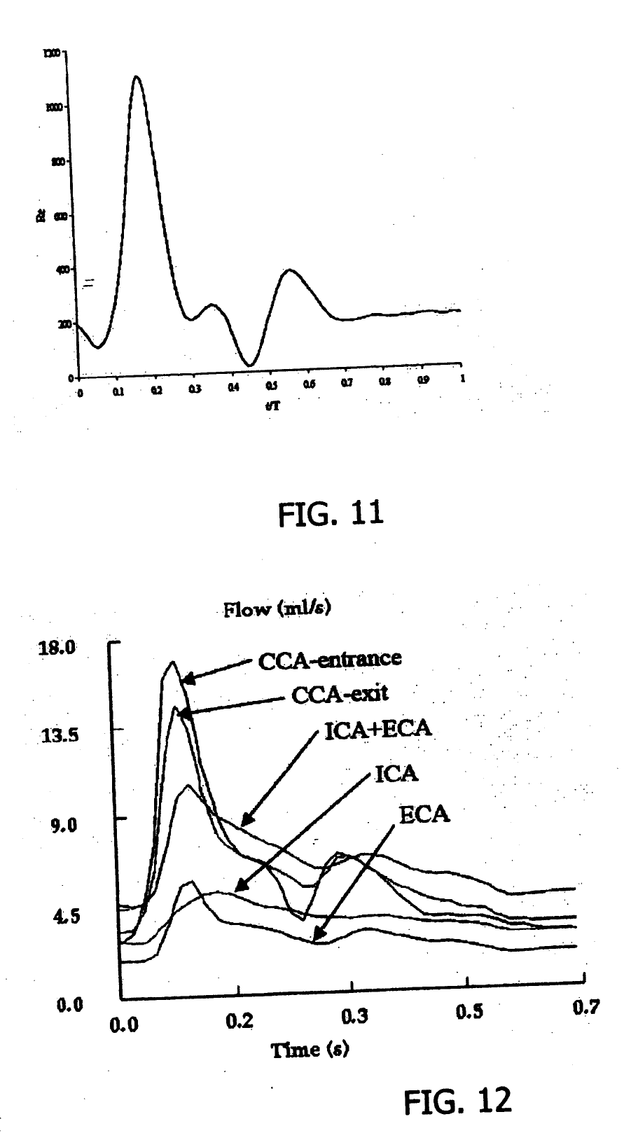

FIGS. 11 and 12 show graphs of the pulsatile flow behavior of the carotid artery. Reynolds number, Re, reflects flow turbulence. FIG. 12 show the volumetric flow rates through various sections of the carotid artery, where CCA is the common carotid artery, the ICA is the internal carotid artery, and ECA is the external carotid artery.

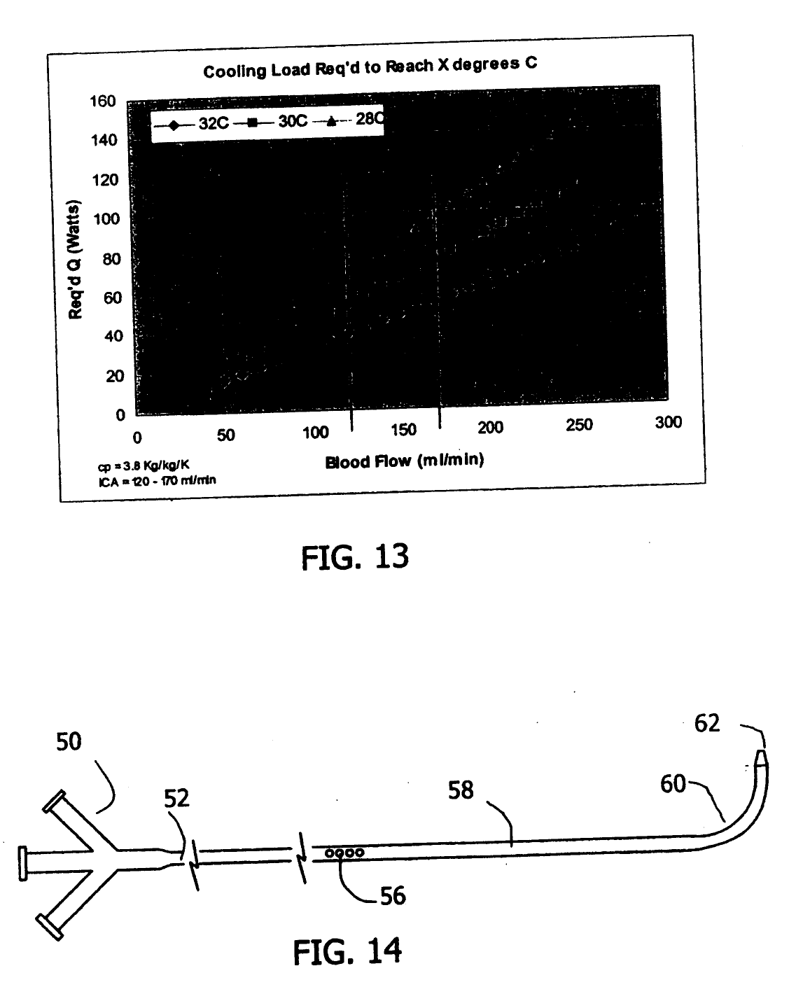

FIG. 13 shows a graph of the required cooling capacities needed to cool various amounts of blood to 32C, 30C, and 28C. This graph defines the required cooling capacity for small artery coolers. It also reveals that small artery cooling can require significantly less cooling capacities than what is required to cool the entire body. The vertical lines at 120 ml/min and 170 ml/min reflect the range of normal blood flow within the internal carotid artery.

Description and Operation FIG. 14-49

FIG. 14 shows a drawing of a blood cooling catheter or device. At the proximal end of the cooling catheter, the end that is outside the patient's body, an interconnection hub 50 allows for coolant flow interconnections and adjunctive therapies. A proximal shaft 52 extends to blood entry holes 56. The entry holes 56 allow blood to enter the interior of the cooling catheter. The blood flows along a distal shaft 58 to a flexible distal section 60 where it exits out a distal tip 62. Selection for the location of the blood entry holes 56 is important to cooling catheter performance. If the holes are too far from the distal tip 62, little or nor blood flow will occur. If the holes are two close to the distal tip 62, there is insufficient surface area for the heat exchange. An optimal distance for these holes is between 20 and 40 cm from the distal tip depending upon cooling catheter geometry and blood flow differential pressures shown in FIGS. 7 and 9.

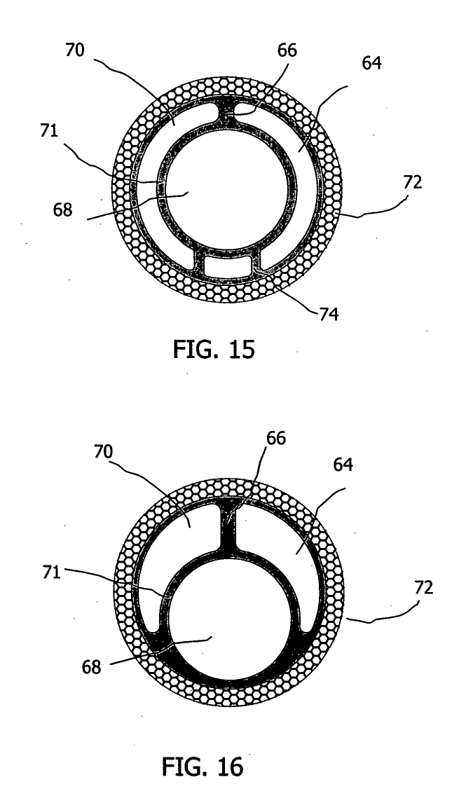

FIGS. 15 and 16 show a cross-section or profile of the cooling catheter. An outer wall 72 of the cooling catheter is a composite material made of polymer and braiding. The typical polymers include polyethylene, polyurethane, and polyether blocked amide. The braiding is made out of stainless steel or a mono-filament fiber. Typical braid thicknesses are around 0.025-0.075 mm. The composite out wall 72 has thickness of 0.18 mm to 0.27 mm. This outer wall makeup is standard in nearly all guide catheters and significantly improves catheter flexural stiffness and longitudinal stiffness, allowing physicians to more easily place the catheter inside the patient. An inner core 71 is a multilumen extrusion made of a lubricious material such as polytetrafluoroethylene (PTFE or Teflon). The inner core provides pathways for both coolant flow and blood flow and generally requires optimization for the cooling catheter to work. An entering coolant pathway 70 and an exiting coolant pathway 64 allow coolant to be circulated through out the cooling catheter. A septum 66 is used to separate the two pathways. An inner blood lumen 68 allows for blood flow and adjunctive therapies, such as angioplasty and stent placement. An additional lumen 74 (FIG. 15) is used to provide an open air pocket for insulation and a space to insert blood entry holes 56. Air has one-tenth the thermal conductivity of PTFE. FIG. 15 inner core geometry offers an advantage in terms of heat transfer effectives since the blood lumen 68 is nearly completely surrounded by either coolant flow (inside 70 or 64) or by insulated lumens (74). The coolant lumens 70 and 64 are quite thin, about 0.2 mm in thickness, potentially creating a flow limiting resistance for the coolant. FIG. 16 geometry offers an advantage for assembly and fabrication as there are only three lumens (70, 64, and 68) and a simpler pathway for the blood entry holes 56.

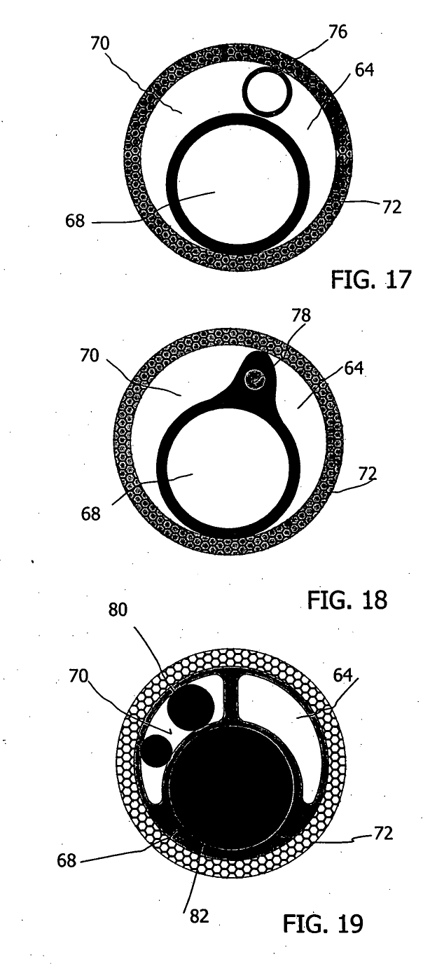

FIGS. 17 and 18 show alternative profiles for the cooling catheter. A separation tube 76 is used to separate coolant inlet 70 and exit pathways 64. In addition the lumen inside the separation tube 76 may be used to carry infused fluid or temperature sensing devices such as thin wire thermocouples 78 (FIG. 18). FIG. 18 avoids the two-tube insertion with a single non-circular extrusion. The pressure difference between inlet coolant flow and outlet coolant flow effectively seals and separates the two streams of flow.

FIG. 19 shows a drawing of how round support mandrels 80 and 82 are used to fabricate the cooling catheter. During the fabrication process braiding is wound around the inner core 71 and radial forces are applied. Additional radial forces are applied as the composite outer wall is created by fusing a thermoplastic polymer into the braid using heat. By using support mandrels, the inner core lumens are not crushed during this fabrication process. Other lumen support methods include high-boiling point liquids, fine grain sand, and non-circular geometry support mandrels.

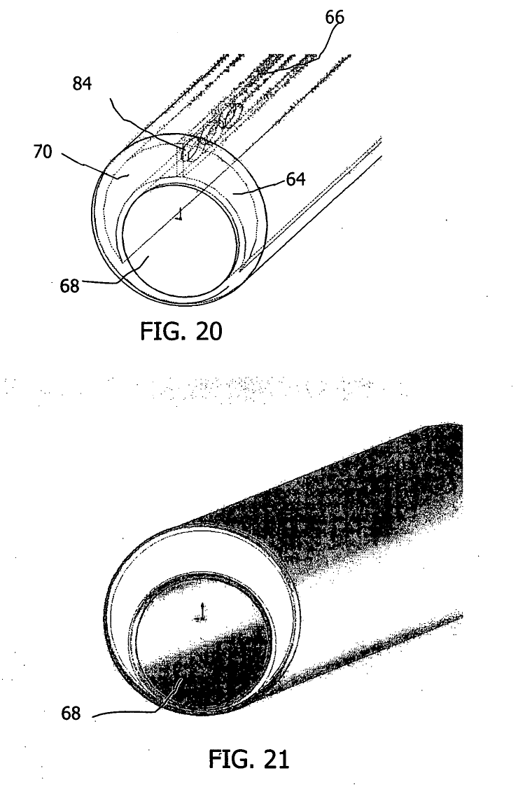

FIG. 20 shows coolant turn-around holes 84 at the distal tip 60 of the cooling catheter. These holes allow the coolant to travel the entire length of the cooling catheter and return back to the proximal hub. To create these holes, drills, end-mills, or surgical scissors may be used to create pathways through the septum 66.

FIG. 21 shows the distal tip 62 of the cooling catheter. Using an adhesive or heat shrink tubing the coolant pathways 70 and 64 (FIG. 20) are sealed. This tip is also smooth and radiopaque to avoid inner vascular wall damage and to allow visualization using fluoroscopy, a standard procedure during cardiovascular intervention.

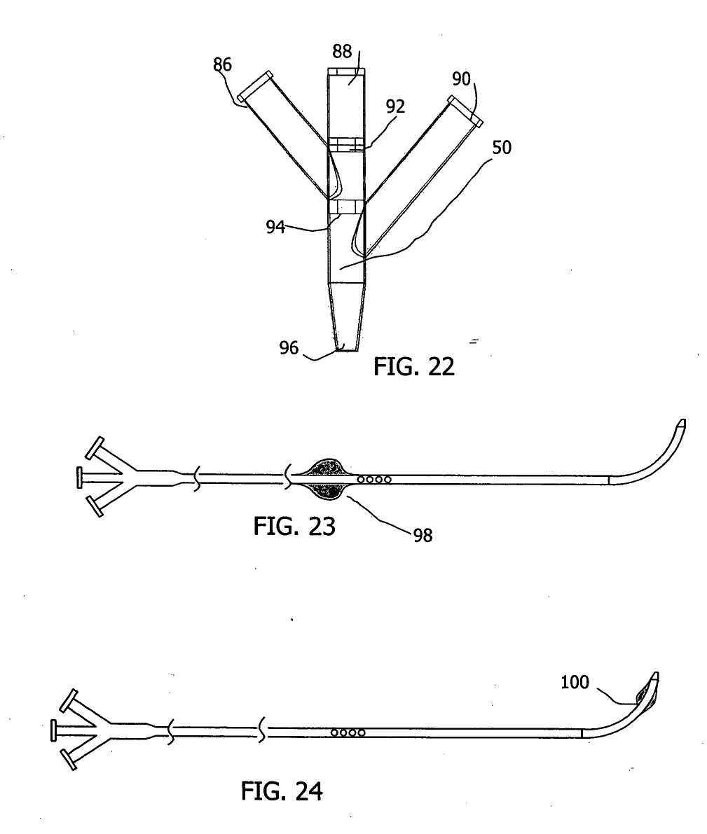

FIG. 22 shows an in-depth drawing of the proximal hub 50. Coolant enters through connection 90 and exits through connection 86. Connection 88 is used to allow other interventional tools to pass through the blood lumen 68 (FIG. 21). Flow separators, 92 and 94, isolate the individual flows of coolant in, coolant out, and interventional tools. These flow separators can be created by using molds, machined collars, or adhesives. A transition section 96 from the hub 50 to the proximal shaft ensures that blood and coolant do not leak. To allow for coolant passage from the hub connectors 90 and 86 into the inner core 71 small holes with a diameter of 1 to 2 mm are drilled into the proximal end of the cooling catheter.

FIGS. 23 and 24 show balloons used to boost blood flow rates inside the cooling catheter. A proximal balloon 98 in FIG. 23 attempts to partially occlude the descending aorta and increase the upstream flow-motivating pressure. A distal balloon 100 in FIG. 24 attempts to partially occlude the ostium to reduce the downstream pressure, increasing the differential pressure along the blood inner lumen 68 (FIG. 15). Both types of balloons may be manufactured using standard dip molding or blow molding techniques. Both types of balloons may be made of standard medical balloon materials such as polyurethane, latex, silicon, nylon or polyethylene terephthalate. The proximal balloon diameter would range in size from 3-6 mm. The proximal balloon length would range in size from 2-5 cm, while the distal balloon length would range in size from 0.5 cm to 3 cm.

The cooling catheter in one preferred embodiment (FIG. 15) can be used in the same fashion that a standard guide catheter is used. Using introducer sheaths (not shown) and fluoroscopy systems (not shown) the cooling catheter is first placed in the ostium of the coronary artery of interest (FIG. 6). Once the catheter has engaged the ostium, natural differential pressure auto-perfuses the catheter and blood travels from the blood entry holes 56 to the distal tip 62. The amount of blood flow is proportional to four factors: the blood viscosity, the inner lumen diameter 68, the distance the blood must travel within the cooling catheter, and the differential pressure between the aorta and the ostium. Medical research indicates that this differential pressure can range from 3-50 mmHg. With inner lumen diameters of 1.2 to 1.8 mm, these natural differential pressures enable blood flow rates ranging from 20 to 100 ml/min when blood entry holes are 20-40 cm from the distal tip 62.

To cool the blood that enters and travels inside the cooling catheter, cooled surfaces are required. The surfaces that come in contact with the coolant flow pathways are cooled. These surfaces draw heat away from the blood as it travels inside the cooling catheter. The coolant is circulated through the coolant pathways using pumps, heat exchangers, and/or chillers (all not shown). Coolant pumps, located in a console (not shown) outside the patient, pump coolant in a closed loop between a warm patient and a cold heat exchanger. The heat exchanger can be made cold by using a standard laboratory chiller, such as Thermo Electron Corporation, Portsmouth, N.H. USA, Model M25. To ensure maximum cooling, the laboratory chiller, using its own internal pump, pumps fluids at 3-4 liters per minute, can use fluids such as propylene glycol mixtures with freeze points as low as −20C. The coolant pump pumps coolant at flow rates ranging from 100-500 ml/min through the heat exchanger. A recirculation loop in the coolant pathway is used to ensure the maximum heat transfer performance of the heat exchanger. So while the catheter coolant flow rates are lower 30-150 ml/min, the heat exchanger receives much higher flows to minimize the coolant-side heat transfer resistances. In the end, this recirculation loop enhances cooling catheter performance by minimizing the coolant entry temperature and maximizing the temperature difference between the cooling catheter and the blood.

With the cooling catheter in place and coolant pumping within it, blood exits from the distal tip 62 of the catheter and enters the organ of interest, primarily the heart or brain. As this is occurring a physician may use the inner lumen or interventional pathway 68 (FIG. 21) to carry out other interventional tasks as needed. This cooling catheter design feature allows the physician to make one arterial penetration to reduce ischemic tissue damage and carry out other interventional work, such as angioplasty and stent placement. Upon completing the interventional procedure of interest, the physician can either remove the catheter or leave it in place to maintain cooled blood perfusion for extended periods of hypothermia tissue protection.

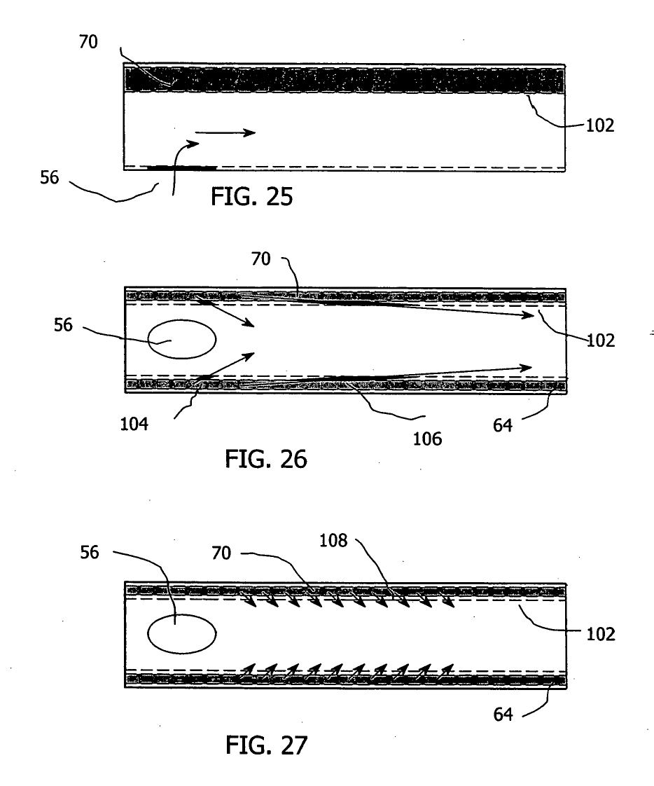

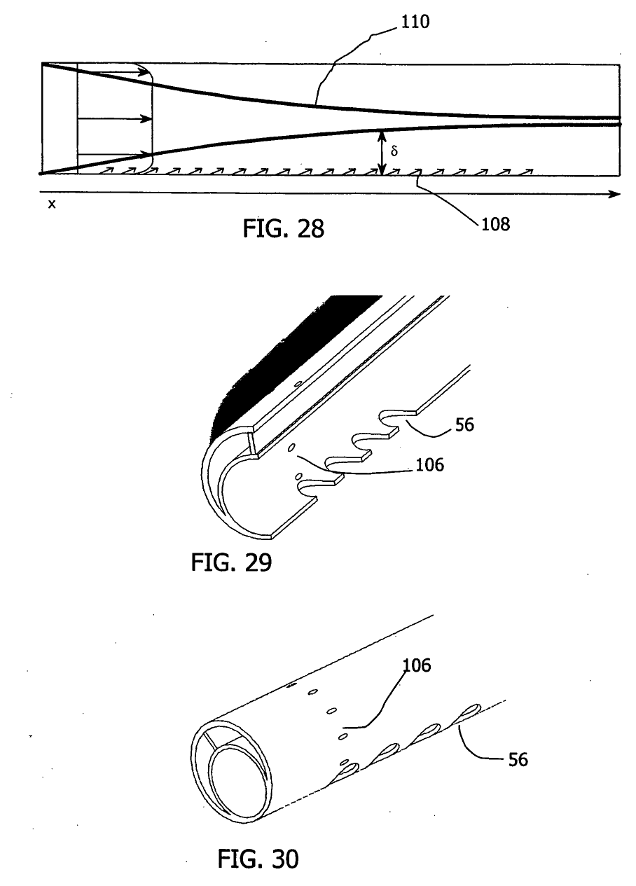

In addition to cold surfaces, cold infusion may also be used to reduce blood temperature in the cooling catheter. This cooling catheter is a hybrid device that combines surface cooling and infusion cooling. FIG. 25 shows a cross-section of the cooling catheter near the blood entry holes 56. Blood enters through these holes and travels towards the lower pressure distal end 62. Along the way the blood is cooled along a cooled inner surface wall 102. FIG. 26 is a drawing of a cooling catheter with coolant infusion, using coolant entry holes 106 and 104. Coolant passes through these holes because of the pressure difference between the coolant pumping pressure and the internal blood pressure. These coolant holes may vary in terms of hole size, angle of penetration, number of holes, and hole array configuration. For example, FIG. 27 shows an array of angle coolant entry holes 108. FIG. 28 shows these holes in relation to a developing hydrodynamic boundary layer.

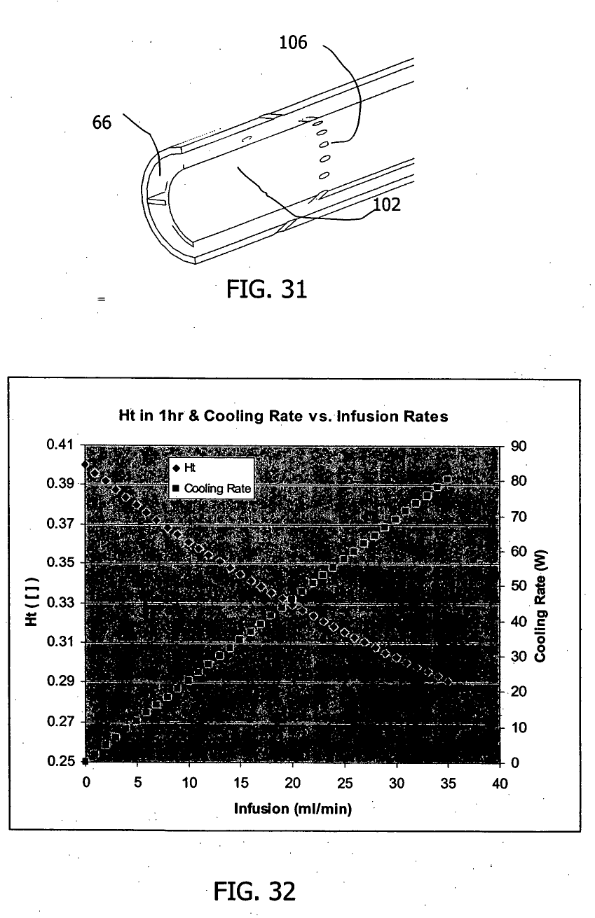

These holes can be made in several different ways: 1) micro drill bit and end mills ranging in size from 0.1 to 0.2 mm, 2) heated needles, and 3) eximer lasers. Eximer laser for example can make holes down to the micrometer level, 1/1000 of a millimeter. In the case of micro drill bits, holes are drilled through the out wall of the inner core 71 (FIG. 14) and sealed with adhesive. FIGS. 29 and 30 show isometric drawings of coolant entry holes near the blood entry holes. FIG. 31 shows these coolant entry holes penetrating from the septum 66 location and into the blood lumen 68.

Coolant infusion enhances cooling catheter performance in two ways, by mixing and exchanging momentum. The first benefit from mixing occurs when the coolant mixes with the blood and comes to an equilibrium temperature. If the coolant infusion rates amount are small relative to the blood flow rates and the coolant enters at 4C, each ml/min of infusion has the potential to cool the blood flowing inside the cooling catheter by approximately 2.3 Watts. The second benefit from mixing is reduced viscosity. This reduction in viscosity enables greater flows inside the blood lumen 68 for equivalent diameters, lengths, and pressure differences. If the coolant chosen is saline (0.9% sodium chloride), a typical infusion fluid, the viscosity is approximately one-third that of blood and mixing dramatically reduces the bulk fluid viscosity. Finally, the entering infusion is angled in the direction of blood flow enabling momentum exchange towards the distal tip of the cooling catheter.

FIG. 32 shows the effect of coolant infusion on hematocrit levels and cooling capacity. In this graph it is assumed that the original hematocrit is 0.4 and the total volume of blood prior to infusion is 5.6 liters. Hematocrit is the volume fraction of blood that is occupied by red blood cells. To avoid unsafe reductions hematocrit a minimum safe level of 0.29 is found in the medical literature. This minimum hematocrit level fixes the maximum infusion rate to a practical limit of 35 ml/min, assuming a 1 hr infusion duration. The cooling catheter is designed to harness the cooling capacity enhancement of infusion without the reducing the hematocrit to unsafe levels.

The operation of this hybrid infusion-surface cooling catheter is nearly identical to the operation of the preferred embodiment described previously. The only difference in operation is the coolant circulation control. To control infusion rates, coolant pumping pressures are monitored with typical pressure transducers (not shown) in the coolant pumping circuit. Furthermore, the amount of infused coolant is directly monitored by either circuit flow meters or coolant reservoir volume monitors.

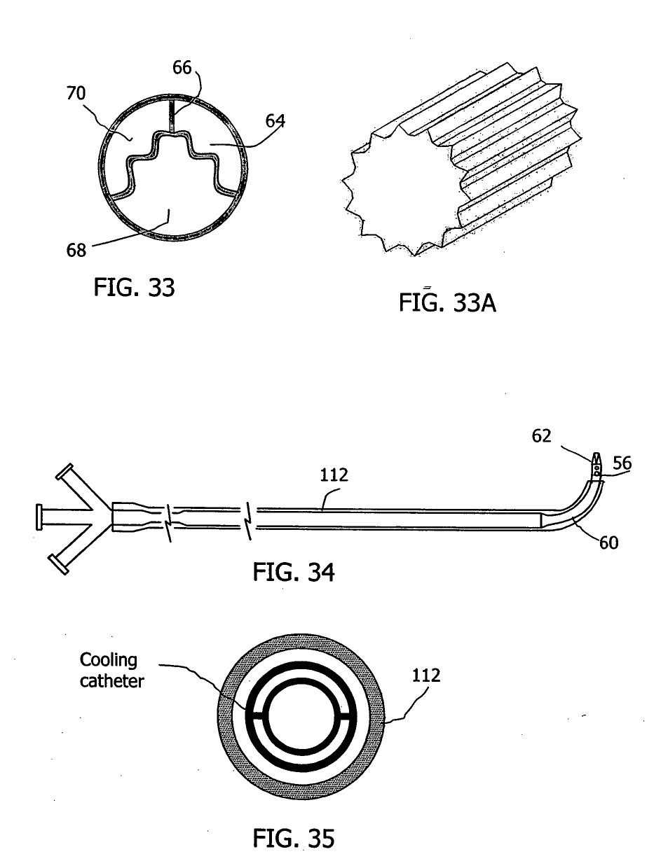

FIG. 33 shows a profile of an inner core extrusion that attempts to maximize the available surface area for coolant to blood heat exchange. Increasing surface area for heat exchange increases the overall cooling capacity of the cooling catheter. This extrusion can be extruded to take this shape or similar shape. Alternatively, this extrusion can be formed through post-processing using mandrels, heat, and radial forces applied with heat shrink tubing. For example, sample indentations on the inner wall 102 can be created by inserting a multi-side mandrel, FIG. 33A and applying the braided wall as described above.

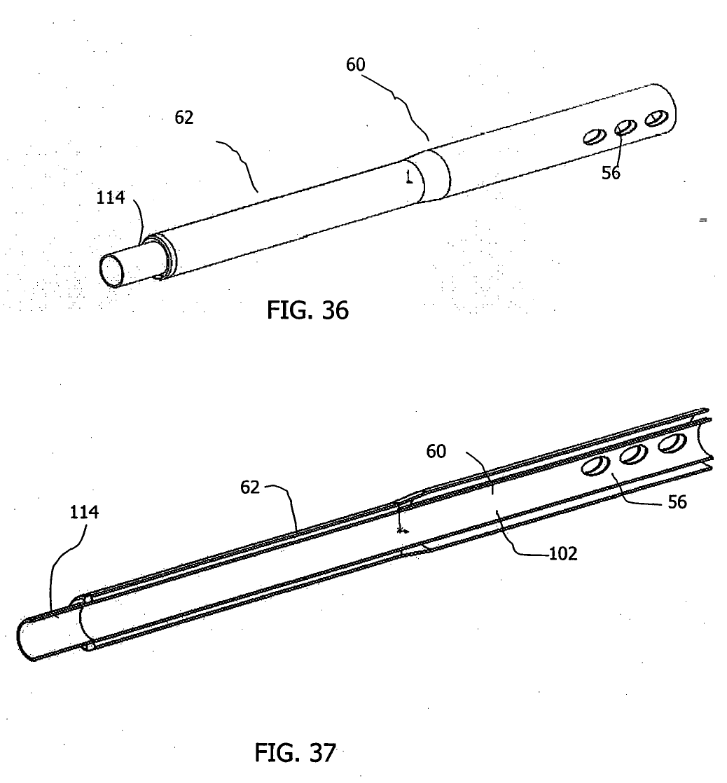

FIGS. 34 and 35 show a cooling catheter that is reduced in scale so that it may pass within a standard guide catheter 112. FIG. 35 shows the profile of the cooling catheter. In this embodiment a braided wall is not necessary since the standard guide catheter has the sufficient properties of longitudinal and torsional stiffness. Blood entry holes 56 in this embodiment are located near the distal tip 62. FIGS. 36 and 37 show isometric views of the distal end 60 of this embodiment. At the distal tip a thin-walled flexible polymer is used such as polyurethane, latex, or silicone 114.

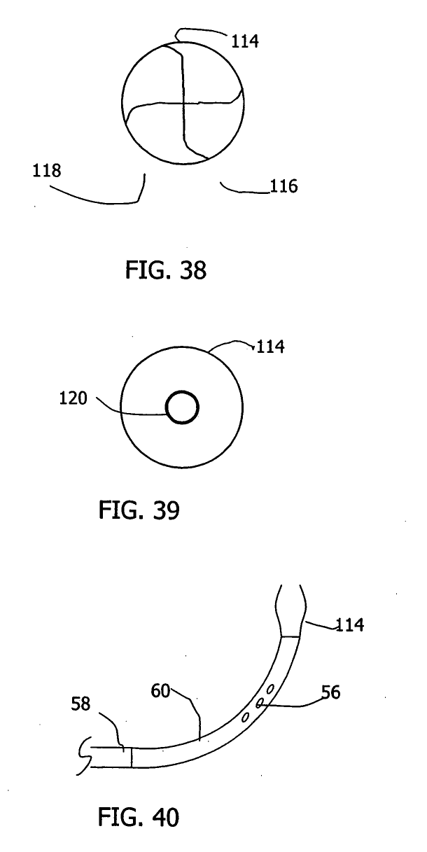

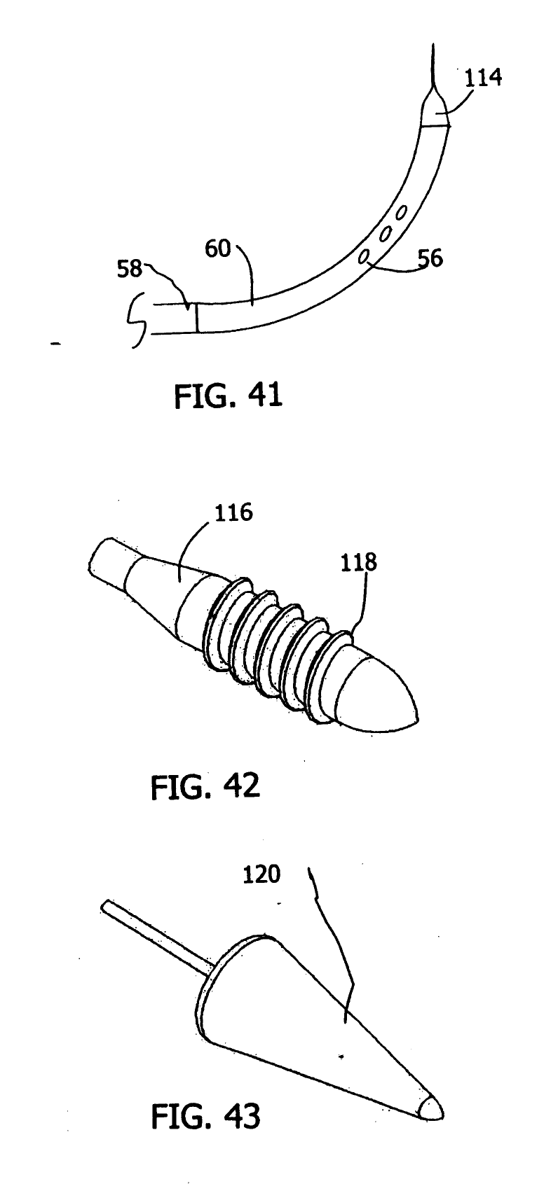

FIGS. 38 and 39 show this tip in two different styles. In FIG. 38 the tip closes like a flow petal when an internal vacuum pressure is applied. In FIG. 39 the tip closes like a funnel when an internal vacuum pressure is applied. FIG. 41 shows how the thin walled polymer tip collapses when an internal vacuum pressure is applied. FIG. 40 shows how the tip expands with an internal positive pressure.

These embodiments use a thin-walled distal tip that enables blood shuttle flow, meaning blood is pulled into and then pushed out of the cooling catheter. There are two steps to the operation of this embodiment. First, a vacuum pressure is applied to the rear hub connected to the inner lumen 88 (FIG. 22). As this vacuum is applied blood is pulled through the blood entry holes 56 and the distal tip 114 is sealed. As blood is pulled into the inner lumen of the cooling catheter it is cooled. In the second step a positive pressure is applied to the hub connection 88 and blood is pushed out largely through the distal tip. This cycling of flow and pressure is achieved by using standard syringe or peristaltic pumps (not shown). The coolant pumping operation for this blood shuttle embodiment does not necessarily change; it operates in a similar fashion as the previously described embodiments.

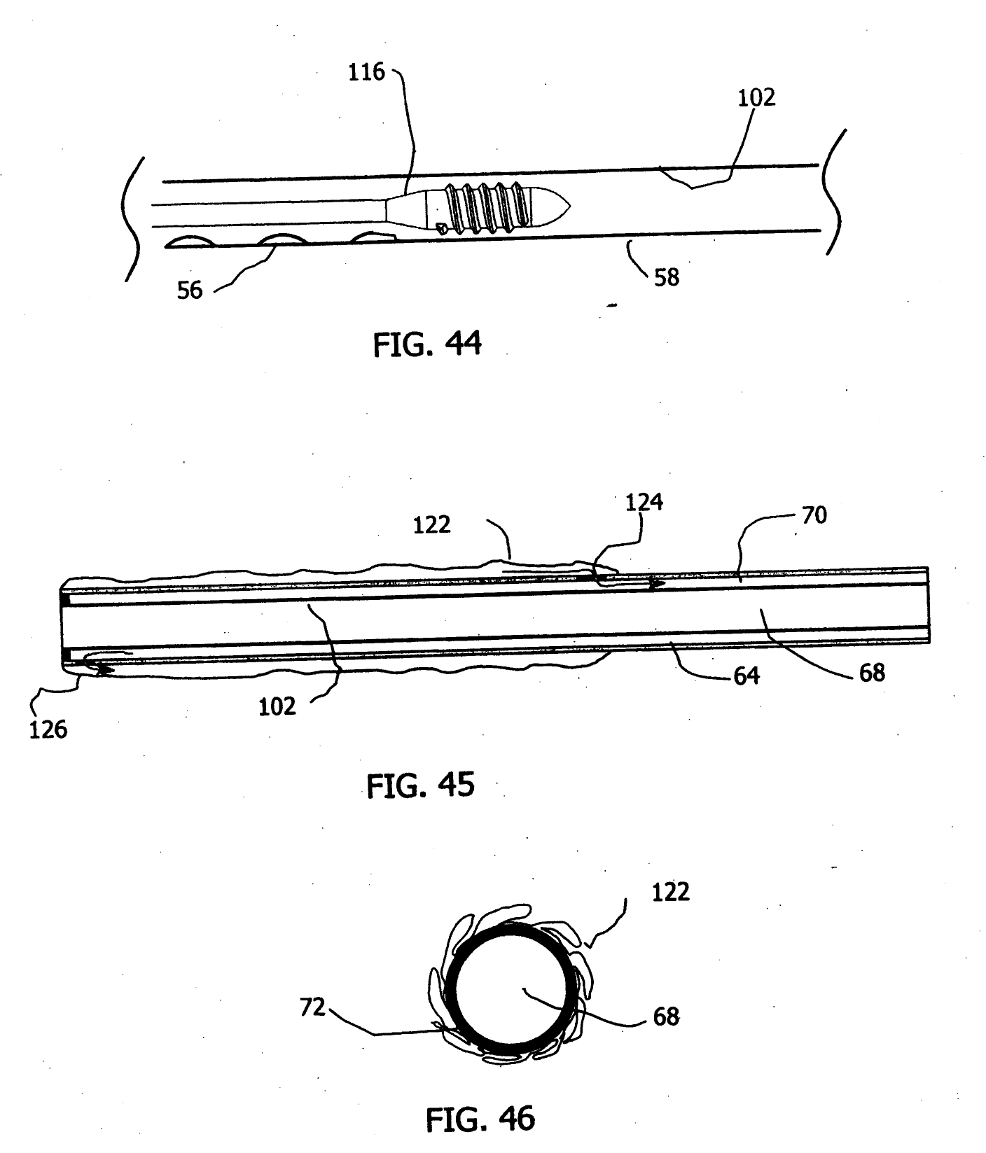

FIGS. 42 and 43 show another means for motivating internal blood flow through the cooling catheter. In FIG. 42 an inflatable turbine balloon 116 is spun to push blood towards the distal tip of the cooling catheter using its blades 118. In FIG. 43, a wedge balloon 120 is used to accomplish the same end. FIG. 44 shows the turbine balloon located inside the cooling catheter. At the proximal hub connection 88 a blood-tight seal using a touhy-burst fitting is used to allow the entry and motion of these two blood flow motivating devices. The balloons may be made using the same material and techniques as mentioned previously for the proximal 98 and distal 100 balloons mentioned above.

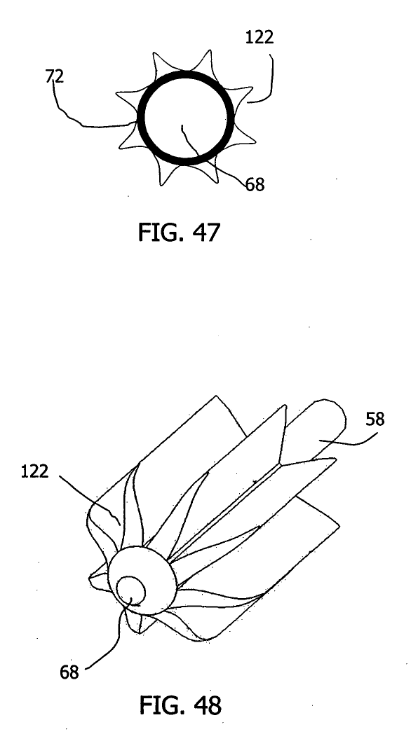

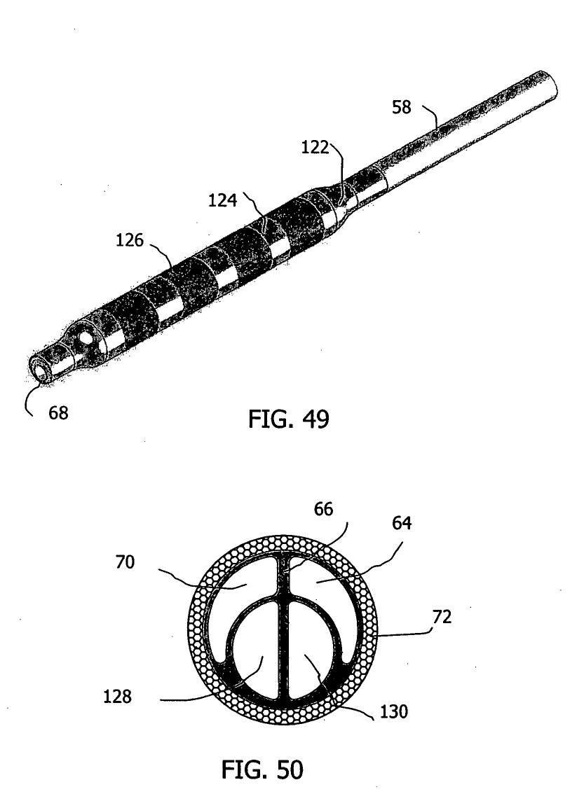

Blood flows in the internal carotid artery at rates of about 150 ml/min as opposed to rates of about 100 ml/min in the left main coronary artery. Based on FIG. 13, this requires greater cooling rates in proportion to the amount of blood flow. In response to this increased cooling need for the brain, combined internal-external cooling catheter embodiments are described. FIG. 45 shows an internal-external cooling catheter. In addition to the typical internal surface area for cooling 102, it provides secondary cooling via an inflatable polymer 122. An entry hole 126 allows coolant to enter the inflatable polymer, while exit holes allow coolant to exit the polymer. The same coolant inlet 70 and coolant exit pathway 64 are used as in the preferred embodiment. FIG. 46 shows the inflatable polymer prior to unfurling with coolant flow initiation. FIG. 47 shows the inflatable polymer unfurled with coolant flow. FIG. 48 shows an isometric view of this unfurled polymer. FIG. 49 shows a 3D view of internal-external cooling catheter that uses active mixing to augment heat transfer. In addition to the additional heat transfer surface area from the flexible polymer, it uses a heterogeneously compliant polymer (more compliant 126 and less compliant 124) that pulsates with internal coolant pressure pulsations. This technology is described in commonly-owned and assigned patent application Ser. No. 10/620,212, entitled Active Mixing Exchange Catheter and Method, which is incorporated by reference.

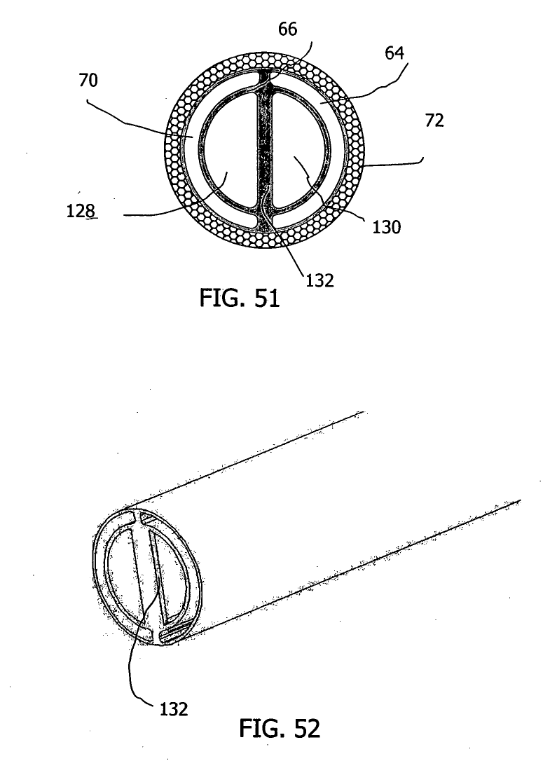

An additional embodiment is shown in FIGS. 50 and 51. In this embodiment the coolant paths have not changed, however the blood flow path has changed. In these embodiments, blood flows in two paths instead of one, 128 and 130. The septum 66 extends from the coolant pathways into the blood pathway. FIG. 52 shows an isometric drawing of the inner core at the distal tip of the cooling catheter. Similar to the previous embodiments this core is made of a lubricious polymer like Teflon. At the distal tip a fluid flow deflection wedge 132 is used to insure fluids from blood flow paths 128 and 130 do not mix during blood ejection and withdrawl.

To operate the embodiments shown in FIGS. 50-52, asynchronous blood shuttling is used. In an identical fashion as previously stated blood is pulled and pushed via an external pump (not shown) connected to the proximal hub 88. However, in this case two separate pumps are use to pull and push blood along cold interior surfaces 102 in an asynchronous fashion. The asynchronicity ensures cool blood is continuously flow to the organ of interest. The wedged 132 improves the efficiency of the design by avoiding pulling in blood that has already been cooled. The coolant operation in this embodiment is similar to the operation mentioned in the previously described embodiments.

In conclusion, the present invention provides, among other things, a system and method for arterial blood or body fluid cooling. Those skilled in the art can readily recognize that numerous variations and substitutions may be made in the invention, its use and its configuration to achieve substantially the same results as achieved by the embodiments described herein. Accordingly, there is no intention to limit the invention to the disclosed exemplary forms. Many variations, modifications and alternative constructions fall within the scope and spirit of the disclosed invention as expressed in the claims.

What is claimed is:

1. A catheter for cooling blood, the catheter comprising:
a tube configured to be inserted into a blood vessel;
a blood-conveyance pathway located inside the tube;
a blood inlet configured to allow blood to enter the blood-conveyance pathway from a blood vessel;
a blood outlet configured to allow blood to move from the blood-conveyance pathway into the blood vessel;
a coolant-supply pathway located inside the tube and adjacent to the blood-conveyance pathway;
a coolant-return pathway located inside the tube; and
a coolant-turn-around connecting the coolant-supply pathway and the coolant-return pathway.

2. The catheter of claim 1, wherein the tube comprises a proximal end and a distal end and wherein the coolant-turn-around is located near the distal end.

3. The catheter of claim 1, wherein the blood inlet comprises a plurality of holes to permit blood flow.

4. The catheter of claim 1, wherein the tube comprises an outer braid.

5. The catheter of claim 1, further comprising an insulator configured to separate at least a substantial portion of the coolant-supply pathway and the coolant-return pathway.

6. The catheter of claim 1, further comprising a hub configured to engage the tube.

7. The catheter of claim 1, further comprising a coolant pump configured to pump coolant through the coolant-supply pathway.

8. The catheter of claim 1, further comprising an infusion inlet configured to enable infusion cooling.

9. The catheter of claim 8, wherein the infusion inlet is configured to enable a portion of a cooling solution in the coolant-supply pathway to pass into the blood-conveyance pathway.

10. The catheter of claim 1, further comprising:
an inflatable sleeve secured around at least a portion of an outside surface of the tube.

11. The catheter of claim 10, wherein the inflatable sleeve comprises a coolant inlet and a coolant outlet.

12. The catheter of claim 11, wherein the coolant inlet is configured to receive a coolant solution from the coolant-supply pathway.

13. The catheter of claim 1, further comprising a means for motivating internal blood flow through the blood-conveyance pathway.

14. The catheter of claim 1, further comprising a balloon configured to motivate internal blood flow through the blood-conveyance pathway.

15. The catheter of claim 1, further comprising a pump configured to motivate internal blood flow through the blood-conveyance pathway.

16. The catheter of claim 1, wherein the a blood-conveyance pathway comprises a first blood-conveyance pathway and a second blood-conveyance pathway.

17. The catheter of claim 1, wherein the blood-conveyance pathway is configured to allow an interventional tool to pass.

18. The catheter of claim 1, wherein the blood-conveyance pathway is non-circular, thereby increasing a heat-exchange surface area.

19. A catheter for cooling blood, the catheter comprising:
a blood-conveyance pathway;
a blood inlet configured to allow blood to enter the blood-conveyance pathway from a blood vessel;
a blood outlet configured to allow blood to move from the blood-conveyance pathway into the blood vessel;
a coolant-supply pathway to the blood-conveyance pathway; and
a coolant-return pathway connected to the coolant-supply pathway.

20. The catheter of claim 19, further comprising an infusion inlet configured to enable infusion cooling.

21. The catheter of claim 20, wherein the infusion inlet is configured to enable a portion of a cooling solution in the coolant-supply pathway to pass into the blood-conveyance pathway.

22. The catheter of claim 21, further comprising:
an inflatable sleeve secured around at least a portion of an outer portion of the coolant-supply pathway.

23. The catheter of claim 22, wherein the inflatable sleeve comprises a coolant inlet and a coolant outlet.

24. The catheter of claim 23, wherein the coolant inlet is configured to receive a coolant solution from the coolant-supply pathway.
